(19) United States
(12) Patent Application Publication
(10) Pub. No.: US 2007/0060971 A1
(43) Pub. Date: Mar. 15, 2007

(54) Hepatic device for treatment or glucose detection

(76) Inventors: Ofer Glasberg, Haifa (IL); Tami Harel, Haifa (IL)

Correspondence Address: WOLF, BLOCK, SHORR AND SOLIS-COHEN LLP, 250 PARK AVENUE, 10TH FLOOR, NEW YORK, NY, 10177, US

(21) Appl. No.: 10/561,740

(22) PCT Filed: Jun. 20, 2004
(86) PCT No.: PCT/IL04/00551

§ 371 Date: Aug. 28, 2006

Related U.S. Application Data
(60) Provisional application No. 60/489,208, filed on Jul. 22, 2003.
(60) Provisional application No. 60/488,964, filed on Jul. 21, 2003.

(51) Int. Cl. A61N 1/36

(52) U.S. Cl. 607/40; 607/44; 607/2

(57) ABSTRACT

A method is provided for treating a subject, including applying an electrical current to a hepatic portal vein (22) of the subject, and configuring the current so as to increase glucose uptake by tissue of the subject. A method is also provided, including sensing an electrical signal generated by a portoarterial glucose sensor of a subject, analyzing the signal, and responsive to the analyzing, detecting eating by the subject.

### CROSS-REFERENCES TO RELATED APPLICATIONS

The present patent application claims priority from (a) U.S. Provisional patent application 60/480,208, filed Jun. 20, 2003, entitled, “Hepatic device for treatment, eating detection, and glucose level detection,” and (b) U.S. Provisional patent application 60/488,964, filed Jul. 21, 2003, entitled, “Gastrointestinal methods and apparatus for use in treating disorders and controlling blood sugar.” Both of these applications are assigned to the assignee of the present application and are incorporated herein by reference.

The present patent application is related to a PCT application filed on even date herewith, entitled, “Gastrointestinal methods and apparatus for use in treating disorders,” which is assigned to the assignee of the present application and is incorporated herein by reference.

### FIELD OF THE INVENTION

The present invention relates generally to electrical stimulation and sensing, and specifically to invasive techniques and apparatus for electrical stimulating and sensing.

### BACKGROUND OF THE INVENTION

The body uses physiological sensors for determining concentrations of circulating substances, such as glucose. For example, it is well known that pancreatic beta cells possess a glucose sensor, which reacts to increases in blood glucose, in order to trigger insulin secretion. More recently, several researchers have identified a glucose sensor that is triggered by a glucose gradient between the hepatic portal vein and the hepatic artery (see, for example, the articles by Burcelin et al., cited hereinbelow). This portoarterial glucose sensor is believed to be connected through afferent hepatic branches of the vagus nerve to glucose-sensitive neurons in the lateral hypothalamus and in the nucleus of the solitary tract. Activation of the portoarterial glucose sensor, such as by food intake, stimulates hepatic glucose uptake, inhibits food intake, inhibits counterregulation induced by hypoglycemic agents, and stimulates glucose utilization by some insulin-sensitive tissues, such as muscle tissue. Some published articles indicate that these processes appear not to be mediated by insulin.

U.S. Pat. No. 6,600,953 to Flesler et al., which is assigned to the assignee of the present patent application and is incorporated herein by reference, describes apparatus for treating a condition such as obesity. The apparatus includes a set of one or more electrodes, which are adapted to be applied to one or more respective sites in a vicinity of a body of a stomach of a patient. A control unit is adapted to drive the electrode set to apply to the body of the stomach a signal, configured such that application thereof increases a level of contraction of muscle tissue of the body of the stomach, and decreases a cross-sectional area of a portion of the body of the stomach for a substantially continuous period greater than about 3 seconds.

PCT Publication WO 02/082968 to Policker et al., which is assigned to the assignee of the present application and is incorporated herein by reference, describes a diet evaluation gastric apparatus, which detects when a patient swallows, and detects the type and amount of matter ingested. The apparatus includes electrodes adapted to be coupled to the fundus and antrum of the patient and to measure electrical and mechanical activity therein, and a control unit to analyze such electrical and mechanical activity and optionally apply electrical energy to modify the activity of tissue of the patient.

US Patent Application Publication 2003/0055464 and PCT Publication WO 01/66183 to Darvish et al., which are assigned to the assignee of the present application and are incorporated herein by reference, describe a pancreatic controller comprising at least one electrode adapted for electrifying at least a portion of a pancreas, and a controller programmed to electrify the electrode so as to positively control at least the effect of at least two members of a group consisting of blood glucose level, blood insulin level and blood level of another pancreatic hormone. In one example, the controller controls insulin, glucagon and/or glucose blood levels.

PCT Publication WO 04/021858 to Harel et al., which is assigned to the assignee of the present application and is incorporated herein by reference, describes a method for glucose level control, including applying an electric field to the pancreas such that blood glucose levels are significantly reduced and blood insulin levels are not significantly increased.

US Patent Application Publication 2004/0059393 to Policker et al., which is assigned to the assignee of the present application and is incorporated herein by reference, describes a method for treating a subject, including receiving a sensor signal responsive to the subject eating, analyzing the sensor signal, and driving a current into tissue of the subject responsive to analyzing the signal.

U.S. Pat. No. 5,231,988 to Wernicke et al., which is incorporated herein by reference, describes techniques for treating and controlling diabetes and other systemic pancreatic endocrine disorders attributable to abnormal levels of secretion of endogenous insulin. An electrical stimulator implanted into or worn external to the patient's body is adapted, when activated, to generate a programmable electrical waveform for application to electrodes implanted on the vagus nerve of the patient. The electrical waveform is programmed using parameter values selected to stimulate or inhibit the vagus nerve to modulate the electrical activity thereof to increase or decrease secretion of natural insulin by the patient's pancreas. The stimulator is selectively activated manually by the patient in response to direct measurement of blood glucose or symptoms, or is activated automatically by programing the activation to occur at predetermined times and for predetermined intervals during the circadian cycle of the patient. Alternatively, the automatic activation is achieved using an implanted sensor to detect the blood glucose concentration, and is triggered when the patient's blood glucose concentration exceeds or falls below a predetermined level depending on whether diabetes or hypoglycemia is being treated.

U.S. Pat. Nos. 5,188,104 and 5,263,480 to Wernicke et al., which are incorporated herein by reference, describe a method for stimulating the vagus nerve of a patient so as to alleviate an eating disorder.

U.S. Pat. No. 5,561,165 to Lautt et al., which is incorporated herein by reference, describes a method for increasing insulin responsiveness and improving glucose tolerance in a mammal, comprising administration of an effective amount of a cholinergic agonist. Pharmaceutical compositions are also described.

PCT Publication WO 01/76690 to Chen et al., which is incorporated herein by reference, describes a method for regulating gastrointestinal action in a subject using a stimulatory electrode and a sensor to provide retrograde feedback control of electrical stimulation to the GI tract. The publication also describes a method for reducing weight in a subject, again using a stimulatory electrode and a sensor to provide retrograde feedback control of electrical stimulation to the stomach. The publication further describes a method for stimulating the vagus nerve of a subject.

PCT Publication WO 02/04068 to Barrett et al., which is incorporated herein by reference, describes a method for treating patients for compulsive overeating, including stimulating left and right branches of the patient's vagus nerve simultaneously with electrical pulses in a predetermined sequence of a first period in which pulses are applied continuously, alternating with a second period in which no pulses are applied. The electrical pulses are preferably applied to the vagus nerve at a supradiaphragmatic location.

U.S. Pat. No. 4,592,339 to Kuzmak et al., which is incorporated herein by reference, describes a gastric band for forming a stoma opening in a stomach for treating morbid obesity. The band is invasively placed around the stomach, and an expandable portion of the band is used to adjust the diameter of the stoma opening.

U.S. Pat. Nos. 5,449,368, 5,226,429, and 5,074,868 to Kuzmak, which are incorporated herein by reference, describe adjustable gastric bands. The size of the stoma opening of the bands can be adjusted by injecting into or removing fluid from an expandable section of the gastric bands.

U.S. Pat. No. 5,938,669 to Klaiber et al., which is incorporated herein by reference, describes an adjustable gastric band for contracting a patient's stomach in order to fight obesity. A gastric band of a known type, implanted around the stomach and including a cavity filled with liquid, is connected by a tube to a control box and a balancing reservoir which are implanted under the patient's skin. The box contains an electric pump and an electronic control unit capable of communicating by radio with a monitor carried by the patient and with a controller intended for the doctor. The controller can operate the pump by remote control to transfer determined volumes of liquid in a closed circuit from the gastric band to the reservoir or vice versa, to adjust the diameter of a passage in the stomach. The monitor receives and signals alarms from the control box.

U.S. Pat. No. 6,067,991 to Forsell, which is incorporated herein by reference, describes an adjustable gastric band including an elongated non-inflatable restriction member, a forming device for forming the restriction member into at least a substantially closed loop around the stomach or the esophagus to define a restriction opening, and a post-operation non-invasive adjustment device for mechanically adjusting the restriction member in the loop to change the size of the restriction opening.

U.S. Pat. No. 6,210,347 to Forsell, which is incorporated herein by reference, describes a food intake restriction device for forming a stoma opening in the stomach or esophagus of a patient.

US Patent to Forsell, which is incorporated herein by reference, describes a food intake restriction device for forming a stoma opening in the stomach or esophagus of a patient. The device comprises an elongated restriction member forming an expandable and contractible cavity formed into an at least substantially closed loop defining a restriction opening, the size of which is reduced upon expansion of the cavity and increased upon contraction of said cavity. A reservoir containing a predetermined amount of hydraulic fluid and connected to the cavity of the restriction member, and a hydraulic operation device for distributing fluid from the reservoir to the cavity to expand the cavity and for distributing fluid from the cavity to the reservoir to contract the cavity, are also implanted in a patient with morbid obesity and operated from outside the patient's body in a non-invasive manner.

U.S. Pat. No. 6,453,907 to Forsell, which is incorporated herein by reference, describes an adjustable gastric band that includes an energy transmission device for wireless transmission of energy of a first form from outside the patient's body.

U.S. Pat. No. 6,454,699 to Forsell, which is incorporated herein by reference, describes a food intake restriction apparatus that includes a restriction device implanted in a patient, which engages the stomach or esophagus to form an upper pouch and a restricted stoma opening in the stomach or esophagus. The restriction device optionally includes at least one implanted sensor for sensing at least one physical parameter of the patient, in which case the control device may control the restriction device in response to signals from the sensor.

US Patent Application Publication 2003/0066536 to Forsell, which is incorporated herein by reference, describes food intake restriction apparatus, including an operable restriction device implanted in a patient and engaging the stomach or esophagus to form a restricted stoma opening in the stomach or esophagus. The apparatus includes a source of energy for energizing the restriction device, and a control device for releasing energy from the source of energy from outside the patient's body. The released energy is used in connection with the operation of the restriction device to vary the size of the stoma opening to allow or substantially prevent the passage of food therethrough. The restriction apparatus optionally includes a pressure sensor for directly or indirectly sensing the pressure in the stomach. The control device may control the restriction device in response to signals from the pressure sensor.

US Patent Application Publication 2001/0011543 to Forsell, which is incorporated herein by reference, describes apparatus for treating morbid obesity or heartburn and reflux disease, including an elongated restriction member formed in a substantially closed loop around a human's stomach or esophagus to form a stoma opening in the stomach or esophagus. The size of the stoma opening is adjustable by an implanted adjustment device. A control device is utilized to control the adjustment device, in order to either reduce or enlarge the size of the stoma opening, for example in response to the time of the day. A sensor, such as a pressure or position sensor, is surgically implanted in the human's body so that the sensor may either directly or indirectly sense a physical parameter of the human, such as the pressure in the stomach or the human's orientation with respect to the horizontal. If in response to sensing by the sensor it is determined by the control device that a significant change in the physical parameter has occurred, then the control device controls the adjustment device to either reduce or enlarge the size of the stoma opening.

U.S. Pat. No. 5,259,399 to Brown, which is incorporated herein by reference, describes a method and apparatus for causing weight loss in obese patients by occupying a segment of the stomach volume using a variable volume bladder filled with fluid. The bladder is inserted into the upper part of the stomach including the fundus through a percutaneous endoscopic gastrostomy tube, which was non-surgically placed to create a permanent channel to the stomach. The inserted bladder is filled and emptied using a filling system for pumping fluid in and out of the bladder according to a predetermined scheme. The filling system comprises a reversible pump, a two-way valve connected to the filling tube, an electronic control means for automatically controlling the action of the filling system, and a battery. The electronic control means is connected to a plurality of sensors placed on the patient's body to detect digestion cycle and hemodynamic parameters. The electronic control means collects information detected by the sensors, governs the filling system according to the obtained information and predetermined operation scheme, and records times and volumes of the fluid transferred through the two-way valve.

U.S. Pat. No. 6,514,718 to Heller et al., which is incorporated herein by reference, describes a small diameter flexible electrode designed for subcutaneous in vivo amperometric monitoring of glucose. The electrode is designed to allow “one-point” in vivo calibration, i.e., to have zero output current at zero glucose concentration, even in the presence of other electroreactive species of serum or blood. The electrode is preferably three or four-layered, with the layers serially deposited within a recess upon the tip of a polyamide insulated gold wire. A first glucose concentration-to-current transducing layer is overcoated with an electrically insulating and glucose flux limiting layer (second layer) on which, optionally, an immobilized interference-eliminating horseradish peroxidase based film is deposited (third layer). An outer (fourth) layer is biocompatible.

U.S. Pat. No. 5,368,028 to Palti, which is incorporated herein by reference, describes systems which utilize implanted chemo-sensitive living cells to monitor tissue or blood concentration levels of chemicals. The implanted cells produce a detectable electrical, optical or chemical signal in response to changes in concentration in surrounding medium. The signal is then detected and interpreted to give a reading indicative of blood concentration levels. Capsules containing chemo-sensitive cells and electrodes for detecting electrical activity are also disclosed.

U.S. Pat. No. 6,544,212 to Galley et al., which is incorporated herein by reference, describes a system for enabling glycemic control, including an insulin delivery unit, a glucose sensor, and a control unit. The control unit includes a processor unit that receives glucose value readings from the glucose sensor, executes an algorithm that predicts a glucose value at a predetermined time in the future, compares that predicted glucose value to a predetermined glucose value range, and determines a corrective amount of insulin to be administered when the predictive glucose value lies outside of the predetermined glucose value range.

U.S. Pat. No. 6,740,072 to Starkweather et al, which is incorporated herein by reference, describes techniques for providing closed loop infusion formulation delivery which calculates a delivery amount based on a sensed biological state by adjusting an algorithm's programmable control parameters. The algorithm calculates a delivery amount having proportional, derivative, and basal rate components. The control parameters may be adjusted in real time to compensate for changes in a sensed biological state that may result from daily events. Safety limits on the delivery amount may be included in the algorithm. The algorithm may be executed by a computing element within a process controller for controlling closed loop infusion formulation delivery. The biological state is sensed by a sensing device which provides a signal to the controller. The controller calculates an infusion formulation delivery amount based on the signal and sends commands to an infusion formulation delivery device which delivers an amount of infusion formulation determined by the commands.

INAMED Corporation (Santa Barbara, Calif.) manufactures and markets the LAP-BAND® System, an FDA-approved adjustable and reversible gastric band for treatment of obesity.

The following articles, which are incorporated herein by reference, may be of interest:

Adachi A et al., “Convergence of hepatoportal glucose-sensitive afferent signals to glucose-sensitive units within the nucleus of the solitary tract,” Neurosci Lett 46:215-218 (1984)

Burcelin R et al., “Portal glucose infusion in the mouse induces hypoglycemia: evidence that the hepatoportal glucose sensor stimulates glucose utilization,” Diabetes 49:1635-1642 (2000)

Burcelin R et al., “Glucose competence of the hepatoportal vein sensor requires the presence of an activated glucagons-like peptide-1 receptor,” Diabetes 50:1720-1728 (2001)

Burcelin R et al., “GLUT4, AMP kinase, but not the insulin receptor, are required for hepatoportal glucose sensor-stimulated muscle glucose utilization,” J. Clin. Invest. 111:1555-1562 (2003)

Burcelin R et al., “Glucose sensing by the hepatoportal sensor is GLUT2-dependent,” Diabetes 49:1643-1648 (2000)

Gardemann A et al., “Control of glucose balance in the perfused rat liver by the parasympathetic innervation,” Biol Chem Hoppe Seyler. 367(7):559-66 (1986)

Gilon P et al., “Mechanisms and physiological significance of the cholinergic control of pancreatic beta-cell function,” Endocr Rev. 22(5):565-604 (2001)

Hartmann H et al., “Direct control of glycogen metabolism in the perfused rat liver by the sympathetic innervation,” Eur J Biochem. 123(3):521-6 (1982)

Johansson B et al., “Static and dynamic components in the vascular myogenic response to passive changes in length as revealed by electrical and mechanical recordings from the rat portal vein,” Circulation Research 36(1):76-83 (1975)

Jungermann K et al., “Regulation of liver metabolism by the hepatic nerves,” Adv Enzyme Regul. 26:63-88 (1987)

Lautt W W et al., “Hepatic parasympathetic neural effect on glucose balance in the intact liver,” Can J Physiol Pharmacol. 56(4):679-82 (1978)

Lee K C, “Reflex suppression and initiation of gastric contractions by electrical stimulation of the hepatic vagus nerve,” Neurosci Lett. 53(1):57-62 (1985)

Martin J R et al., “Loss of glucagon suppression of feeding after vagotomy in rats,” Am J Physiol 234:314-318 (1978)

Niijima A, “Afferent impulse discharges from glucoreceptors in the liver of the guinea pig,” Ann. N.Y. Acad. Sci. 157:690-700 (1969)

Niijima A, “The effect of D-glucose on the firing rate of glucose-sensitive vagal afferents in the liver in comparison with the effect of 2-deoxy-D-glucose,” J Auton Nerv Syst. 10(3-4):255-60 (1984)

Sakaguchi T et al., “Effects of electrical stimulation of the hepatic vagus nerve on the plasma insulin concentration in the rat,” Brain Res 164:314-316 (1979)

Schmitt M, “Influence of hepatic portal receptors on hypothalamic feeding and satiety centers,” Am J Physiol 225:1089-1095 (1973)

Shimazu T et al., “Reciprocal influences of the ventromedial and lateral hypothalamic nuclei on blood glucose level and liver glycogen content,” Nature 210:1178-1179 (1966)

Shimazu T, “Glycogen synthetase activity in liver: regulation by the autonomic nerves,” Science 156(779):1256-7 (1967)

Shimazu T et al., “Regulation of glycogen metabolism in liver by the autonomic nervous system. II. Neural control of glycogenolytic enzymes,” Biochim Biophys Acta. 165(3):335-48 (1968)

Shimazu T et al., “Regulation of glycogen metabolism in liver by the autonomic nervous system. 3. Differential effects of sympathetic-nerve stimulation and of catecholamines on liver phosphorylase,” Biochim Biophys Acta. 165(3):349-56 (1968)

Shimau T et al., “Regulation of glycogen metabolism in liver by the autonomic nervous system. IV. Neural control of glycogen biosynthesis,” Biochim Biophys Acta. 252(1):18-27 (1971)

Shimazu T, “Regulation of glycogen metabolism in liver by the autonomic nervous system. V. Activation of glycogen synthetase by vagal stimulation,” Biochim Biophys Acta. 252(1):28-38 (1971)

Shimazu T et al., “Regulation of glycogen metabolism in liver by the autonomic nervous system. VI. Possible mechanism of phosphorylase activation by the splanchnic nerve,” Biochim Biophys Acta. 385(2):242-56 (1975)

Shimazu T et al., “Hypothalamic control of liver glycogen metabolism in adult and aged rats,” Brain Res. 144(2):343-52 (1978)

Shimazu T, “Nervous control of peripheral metabolism,” Acta Physiol Pol. 30(18 Suppl):1-18 (1979)

Shimazu T, “Central nervous system regulation of liver and adipose tissue metabolism,” Diabetologia. 20 Suppl:343-56 (1981)

Shimazu T, “Reciprocal innervation of the liver: its significance in metabolic control,” Adv Metab Disord. 10:355-84 (1983)

Shimizu N et al., “Functional correlations between lateral hypothalamic glucose-sensitive neurons and hepatic portal glucose-sensitive units in rat,” Brain Res 265:49-54 (1983)

Shimazu T, “Innervation of the liver and glucoregulation: roles of the hypothalamus and autonomic nerves,” Nutrition. 12(1):65-6 (1996)

Watanabe Y et al, “Neural control of biosynthesis and secretion of serum transferrin in perfused rat liver,” Biochem J. 267(2):545-8 (1990)

Yamatani K et al., “Impaired vagus nerve-mediated control of insulin secretion in Wistar fatty rats,” Metabolism. 47(10):1167-73 (1998)

Yoshimatsu H et al., “Lateral and ventromedial hypothalamic influences on hepatic autonomic nerve activity in the rat,” Brain Res Bull. 21(2):239-44 (1988)

### SUMMARY OF THE INVENTION

In some embodiments of the present invention, a method for treating a subject comprises applying an electrical current to the hepatic portal vein. Such stimulation generally increases uptake of blood glucose, e.g., by muscle, liver, and/or adipose tissue, thereby normalizing postprandial hyperglycemia, substantially lowering inter-meal blood glucose levels, and/or reducing food intake of the subject. The current is typically configured to stimulate afferent nerve terminals embedded in the wall of the hepatic portal vein and/or in the liver. For some applications, the current is also configured to minimize artificially-induced activation of muscle tissue of the vein. The current is typically applied using one or more electrodes, which are either coupled to a surface of the hepatic portal vein, or implanted in the vein. Such electrical stimulation therefore is generally useful for treating subjects suffering from various medical conditions, such as obesity, diabetes, heart disease, and/or hypertension, or for preventively treating subjects considered at risk of developing such conditions. For some applications, such stimulation is useful for directly treating diabetes even in the absence of weight loss, i.e., the treatment is not necessarily mediated by weight loss.

In some embodiments of the present invention, a method for detecting eating by a subject comprises sensing a signal generated by a portoarterial glucose sensor of the subject. The signal is typically sensed by sensing electrical activity of the hepatic portal vein indicative of activity of afferent nerve fibers that innervate the vein. Such sensing is typically performed using one or more electrodes, which are either coupled to a surface of the hepatic portal vein, or implanted in the vein. Electrical activity of afferent nerve fibers that innervate the hepatic portal vein is generally correlated with the quantity of glucose recently absorbed by the small intestine during eating, and thus serves as an indicator of eating.

In some embodiments of the present invention, a method for detecting eating by a subject comprises sensing changes in a concentration of a blood constituent, such as blood glucose, carbohydrate, fat, or protein concentration, in the hepatic portal vein. Such changes are generally correlated with the quantity of glucose, carbohydrate, fat, or protein recently absorbed by the small intestine during eating. For some applications, such changes are detected by measuring hepatic portal blood flow, which tends to increase corresponding to increases in postprandial portal blood glucose, carbohydrate, fat, or protein levels. The changes in blood flow may be detected using a flow meter attached to the portal vein, such as a pulsed Doppler ultrasonic flow meter, or a meter that utilizes the thermodilution principle. Alternatively, the changes in blood flow are measured by measuring changes in impedance between electrodes placed on the external surface of the portal vein. For some applications, the changes in the concentration of the blood constituent are measured using chemical or non-chemical blood analysis techniques, such as: (a) near-infrared or infrared absorption spectroscopy, or (b) a laser transducer implanted on the portal vein together with an acoustic sensor that measures changes in acoustic reflections from the blood, which reflections are correlated with blood glucose level.

In some embodiments of the present invention, hepatic portal vein stimulation is applied responsive to detection of eating by the subject. For some applications, such detection is performed using the techniques described hereinabove for sensing a portoarterial glucose sensor signal and/or sensing hepatic portal blood glucose, carbohydrate, fat, or protein concentrations. Alternatively, such detection is performed using techniques described in the above-mentioned '953 patent and/or '968 PCT publication, or using techniques known in the art. In other embodiments, hepatic portal vein stimulation is applied generally constantly, not responsive to detection of eating. Alternatively, the stimulation is applied periodically, such as during certain times of day or night.

There is therefore provided, in accordance with an embodiment of the present invention, a method for treating a subject, including:

applying an electrical current to a hepatic portal vein of the subject; and

configuring the current so as to increase glucose uptake by tissue of the subject.

There is also provided, in accordance with an embodiment of the present invention, a method for detecting eating by a subject, including:

sensing an electrical signal generated by a portoarterial glucose sensor of the subject; and

analyzing the signal in order to detect the eating.

For some applications, sensing the signal includes sensing electrical activity of a hepatic portal vein of the subject. Alternatively or additionally, sensing the signal includes sensing electrical activity of a hepatic branch of a vagus nerve of the subject.

There is further provided, in accordance with an embodiment of the present invention, a method for detecting eating by a subject, including:

measuring a concentration of glucose, carbohydrate, fat, or protein in a hepatic portal vein of the subject; and

analyzing the concentration in order to detect the eating.

For some applications, measuring the concentration includes measuring a rate of blood flow within the hepatic portal vein. For some applications, analyzing the concentration includes analyzing the concentration in order to detect a composition of food eaten by the subject.

There is still further provided, in accordance with an embodiment of the present invention, a method for treating a subject, including:

applying an electrical current to a hepatic portal vein of the subject; and

configuring the current so as to increase glucose uptake by tissue of the subject.

For some applications, configuring the current includes configuring the current so as to increase the glucose uptake by a physiological mechanism not mediated by insulin. Alternatively or additionally, configuring the current includes configuring the current so as to increase the glucose uptake by a physiological mechanism mediated by insulin.

For some applications, applying the current includes applying the current only at certain predetermined times of the day. For some applications, the method includes receiving a command from the subject, and applying the current includes applying the current responsively to receiving the command.

For some applications, configuring the current includes configuring the current to stimulate afferent nerve terminals embedded in a site selected from the list consisting of: a wall of the portal vein, and a liver of the subject.

For some applications, configuring the current includes configuring the current to minimize artificially-induced activation of muscle tissue of the portal vein.

For some applications, applying the current includes selecting a subject suffering from a condition selected from the list consisting of: obesity, type I diabetes, type II diabetes, heart disease, and hypertension; and applying the current to the portal vein of the selected subject.

For some applications, configuring the current includes configuring the current to have a frequency of between about 0.1 Hz and about 5 Hz. For some applications, configuring the current includes configuring the current to have a frequency of between about 5 Hz and about 100 Hz. For some applications, configuring the current includes configuring the current to have a frequency of between about 100 Hz and about 1 kHz. For some applications, configuring the current includes configuring the current to have an amplitude of between about 0.1 and about 1 milliamps. For some applications, configuring the current includes configuring the current to have an amplitude of between about 1 and about 15 milliamps. For some applications, configuring the current includes configuring the current to have an amplitude of between about 15 and about 20 milliamps.

For some applications, applying the current includes applying the current not responsively to a detection of eating by the subject.

For some applications, applying the current includes applying one or more electrodes to an external surface of the portal vein, and driving the electrodes to apply the current.

For some applications, applying the current includes placing one or more electrodes in the portal vein, and driving the one or more electrodes to apply the current. For some applications, placing the electrodes includes:

making an opening in a mesenteric vein of the subject that empties into a superior mesenteric vein of the subject;

inserting at least one of the electrodes through the opening; and

advancing the at least one of the electrodes through the mesenteric vein and the superior mesenteric vein until a distal region of the at least one of the electrodes reaches the portal vein.

For some applications, applying the current includes applying the current at least intermittently during a period having a duration of at least 3 days. For some applications, applying the current includes applying the current at least intermittently during a period having a duration of at least one month.

For some applications, applying the current includes detecting an indication of a blood glucose concentration of the subject, and, responsive thereto, applying the current. For some applications, applying the current includes detecting an indication of eating by the subject, and, responsive thereto, applying the current. For some applications, detecting the indication includes sensing an electrical signal generated by a portoarterial glucose sensor of the subject; and detecting the indication by analyzing the signal. For some applications, detecting the indication includes measuring an indication of a concentration of at least one blood constituent in the portal vein of the subject, the constituent selected from the list consisting of: glucose, carbohydrate, fat, and protein.

There is additionally provided, in accordance with an embodiment of the present invention, a method including:

sensing an electrical signal generated by a portoarterial glucose sensor of a subject;

analyzing the signal; and

responsive to the analyzing, detecting eating by the subject.

For some applications, the method includes reducing an appetite of the subject responsively to detecting the eating.

For some applications, the method includes:

responsively to detecting the eating, applying an electrical stimulating signal to at least one site selected from the list consisting of: a site in a vicinity of a colon of the subject, and a site in a vicinity of a distal small intestine of the subject; and

configuring the stimulating signal to stimulate L-cells to increase secretion of at least one hormone selected from the list consisting of: glucagon-like-peptide-1 (GLP-1) and peptide YY (PYY).

For some applications, the method includes:

responsively to detecting the eating, applying an electrical inhibiting signal to at least one site selected from the list consisting of: a site in a vicinity of a stomach of the subject, and a site in a vicinity of a duodenum of the subject; and

configuring the inhibiting signal to inhibit secretion of ghrelin.

For some applications, the method includes:

responsively to detecting the eating, applying a vagal electrical signal to a vagus nerve of the subject; and

configuring the vagal signal to stimulate secretion of at least one hormone selected from the list consisting of: glucagon-like-peptide-1 (GLP-1) and peptide YY (PYY)

For some applications, the method includes:

responsively to detecting the eating, applying a vagal electrical signal to a vagus nerve of the subject; and

configuring the vagal signal to inhibit secretion of ghrelin.

For some applications, the method includes applying a pancreas electrical signal to a pancreas of the subject responsively to detecting the eating.

For some applications, analyzing the signal includes determining an indication of a quantity of glucose recently absorbed by a small intestine of the subject during the eating, and detecting the eating includes detecting the eating by analyzing the indication.

In an embodiment, sensing the signal includes sensing electrical activity of a hepatic portal vein of the subject. For some applications, sensing the signal includes applying one or more electrodes to an external surface of the portal vein, and sensing the signal using the electrodes. For some applications, analyzing the signal includes detecting a decrease in the electrical activity of the portal vein. Alternatively or additionally, analyzing the signal includes detecting an increase in the electrical activity of the portal vein. For some applications, analyzing the signal includes detecting changes in heights of different measured peaks of the electrical activity of the portal vein. For some applications, analyzing the signal includes detecting changes in relative timing of different peaks in the electrical activity of the portal vein. For some applications, sensing the electrical activity includes sensing electrical activity of afferent nerve fibers that innervate the portal vein.

For some applications, sensing the signal includes placing one or more electrodes in the portal vein, and sensing the signal using the electrodes. For some applications, placing the electrodes includes:

making an opening in a mesenteric vein of the subject that empties into a superior mesenteric vein of the subject;

inserting at least one of the electrodes through the opening; and

advancing the at least one of the electrodes through the mesenteric vein and the superior mesenteric vein until a distal region of the at least one of the electrodes reaches the portal vein.

In an embodiment, sensing the signal includes sensing electrical activity of a hepatic branch of a vagus nerve of the subject. For some applications, sensing the electrical activity the hepatic branch includes sensing afferent impulses in the hepatic branch generated by the portoarterial glucose sensor.

For some applications, the method includes:

responsively to detecting the eating, applying a vagal electrical signal to the hepatic branch; and

configuring the vagal signal to reduce an appetite of the subject.

For some applications, configuring the vagal signal includes configuring the vagal signal to mimic natural afferent nerve signals generated by the portoarterial glucose sensor.

There is yet additionally provided, in accordance with an embodiment of the present invention, a method for detecting eating by a subject, including:

measuring an indication of a concentration of at least one blood constituent in a hepatic portal vein of the subject, the constituent selected from the list consisting of glucose, carbohydrate, fat, and protein;

analyzing the indication of the concentration; and

responsive to the analyzing, detecting the eating.

For some applications, measuring the indication of the concentration includes measuring the indication of the concentration using a chemical blood analysis technique.

For some applications, analyzing the indication of the concentration includes detecting an indication of a composition of food eaten by the subject, responsive to the analyzing.

For some applications, measuring the indication of the concentration includes measuring the indication of the concentration using a non-chemical blood analysis technique. For some applications, measuring the indication of the concentration includes measuring the indication of the concentration using near-infrared or infrared absorption spectroscopy. For some applications, measuring the indication of the concentration includes measuring acoustic reflections from blood in the portal vein.

In an embodiment, measuring the indication of the concentration includes measuring a rate of blood flow within the hepatic portal vein. For some applications, measuring the rate of blood flow includes measuring the rate of blood flow using a flow meter. Alternatively or additionally, measuring the rate of blood flow includes measuring a change in impedance between electrodes placed on an external surface of the portal vein.

There is also provided, in accordance with an embodiment of the present invention, a method including:

sensing an electrical signal generated by a portoarterial glucose sensor of a subject;

analyzing the signal; and

responsive to the analyzing, detecting an indication of a blood glucose concentration of the subject.

For some applications, the method includes reducing an appetite of the subject responsively to detecting the indication of the blood glucose concentration.

For some applications, the method includes reducing the blood glucose concentration responsively to detecting the indication of the blood glucose concentration.

For some applications, the method includes:

responsively to detecting the indication of the blood glucose concentration, applying an electrical stimulating signal to at least one site selected from the list consisting of: a site in a vicinity of a colon of the subject, and a site in a vicinity of a distal small intestine of the subject; and

configuring the stimulating signal to stimulate L-cells to increase secretion of at least one hormone selected from the list consisting of: glucagon-like-peptide-1 (GLP-1) and peptide YY (PYY).

In an embodiment, the method includes increasing a blood insulin level responsively to detecting the blood glucose concentration. For some applications, increasing the blood insulin level includes supplying insulin to a blood circulation of the subject from an insulin pump. For some applications, increasing the blood insulin level includes applying a pancreatic electrical signal to a pancreas of the subject, and configuring the pancreatic signal to increase insulin secretion. For some applications, increasing the blood insulin level includes applying a vagal electrical signal to a vagus nerve of the subject, and configuring the vagal signal to cause an increase in insulin secretion by a pancreas of the subject.

In an embodiment, sensing the signal includes sensing electrical activity of a hepatic portal vein of the subject. For some applications, analyzing the signal includes detecting a decrease in the electrical activity of the portal vein. For some applications, analyzing the signal includes detecting an increase in the electrical activity of the portal vein. For some applications, analyzing the signal includes detecting changes in heights of different measured peaks of the electrical activity of the portal vein. For some applications, analyzing the signal includes detecting changes in relative timing of different peaks in the electrical activity of the portal vein. For some applications, sensing the electrical activity includes sensing electrical activity of afferent nerve fibers that innervate the portal vein.

For some applications, sensing the signal includes applying one or more electrodes to an external surface of the portal vein, and sensing the signal using the electrodes. For some applications, sensing the signal includes placing one or more electrodes in the portal vein, and sensing the signal using the one or more electrodes. For some applications, placing the electrodes includes:

making an opening in a mesenteric vein of the subject that empties into a superior mesenteric vein of the subject;

inserting at least one of the electrodes through the opening; and

advancing the at least one of the electrodes through the mesenteric vein and the superior mesenteric vein until a distal region of the at least one of the electrodes reaches the portal vein.

In an embodiment, sensing the signal includes sensing electrical activity of a hepatic branch of a vagus nerve of the subject. For some applications, sensing the electrical activity the hepatic branch includes sensing afferent impulses in the hepatic branch generated by the portoarterial glucose sensor.

For some applications, the method includes:

responsively to detecting the indication of the blood glucose concentration, applying a vagal electrical signal to the hepatic branch; and

configuring the vagal signal to reduce an appetite of the subject.

For some applications, configuring the vagal signal includes configuring the vagal signal to mimic natural afferent nerve signals generated by the portoarterial glucose sensor.

There is further provided, in accordance with an embodiment of the present invention, a method for stimulating a vein of a subject, including:

placing, around an external surface of the vein, a wire shaped so as to define an arc of between about 270 and about 359 degrees; and

driving an electrical current through the wire, so as to stimulate the vein.

In an embodiment, the vein includes a hepatic portal vein, and placing the wire includes placing the wire around the external surface of the portal vein.

For some applications, the electrode includes a removable curved needle, and placing the wire includes:

using the needle to draw the electrode around the external surface of the vein beneath at least a portion of connective tissue surrounding the vein; and

removing the needle after completion of the drawing.

There is still further provided, in accordance with an embodiment of the present invention, a method for sensing electrical activity of a vein of a subject, including:

placing, around an external surface of the vein, a wire shaped so as to define an arc of between about 270 and about 359 degrees; and

sensing the electrical activity of the vein, using the wire.

In an embodiment, the vein includes a hepatic portal vein, and placing the wire includes placing the wire around the external surface of the portal vein.

For some applications, the electrode includes a removable curved needle, and placing the wire includes:

using the needle to draw the electrode around the external surface of the vein beneath at least a portion of connective tissue surrounding the vein; and

removing the needle after completion of the drawing.

There is additionally provided, in accordance with an embodiment of the present invention, a method for placing an electrode in a hepatic portal vein of a subject, the method including:

making an opening in a mesenteric vein of the subject that empties into a superior mesenteric vein of the subject;

inserting the electrode through the opening; and

advancing the electrode through the mesenteric vein and the superior mesenteric vein until a distal region of the electrode reaches the portal vein.

For some applications, the method includes securing a proximal end of the electrode to the mesenteric vein in a vicinity of the opening. For some applications, the method includes tying off the mesenteric vein at a site of the mesenteric vein upstream from the opening.

There is yet additionally provided, in accordance with an embodiment of the present invention, apparatus for treating a subject, including:

one or more electrodes, adapted to be applied to a hepatic portal vein of the subject; and

a control unit, adapted to:
drive the electrodes to apply an electrical current to the portal vein, and
configure the current to increase glucose uptake by tissue of the subject.

For some applications, the electrodes are adapted to be applied to an external surface of the portal vein. For some applications, each of the electrodes includes a wire shaped so as to define an arc of between about 270 and about 359 degrees, and the electrodes are adapted to be applied around the external surface of the portal vein.

For some applications, the electrodes are adapted to be placed in the portal vein. For some applications, the electrodes include an electrode lead having a distal region and a proximal end, the electrode lead adapted to be placed in the portal vein by being advanced through: (a) an opening in a mesenteric vein of the subject that empties into a superior mesenteric vein of the subject, (b) the mesenteric vein, and (c) the superior mesenteric vein, until a distal region of the electrode reaches the portal vein.

There is also provided, in accordance with an embodiment of the present invention, apparatus including:

one or more electrodes, adapted to sense an electrical signal generated by a portoarterial glucose sensor of a subject; and

a control unit, adapted to:

analyze the signal, and

responsive to the analysis, detect eating by the subject.

There is further provided, in accordance with an embodiment of the present invention, apparatus for detecting eating by a subject, including:

a food intake detection device, adapted to be coupled to a hepatic portal vein of the subject, and to measure an indication of a concentration of at least one blood constituent in the portal vein, the constituent selected from the list consisting of: glucose, carbohydrate, fat, and protein; and

a control unit, adapted to:
analyze the concentration, and
responsive to the analysis, detect the eating.

For some applications, the food intake detection device includes a chemical blood analysis device.

For some applications, the control unit is adapted to detect an indication of a composition of food eaten by the subject, responsive to the analyzing.

For some applications, the food intake detection device includes a non-chemical blood analysis device. For some applications, the non-chemical blood analysis device is adapted to perform near-infrared or infrared absorption spectroscopy. For some applications, the non-chemical blood analysis device includes a laser transducer, adapted to measure acoustic reflections from blood in the portal vein.

For some applications, the food intake detection device includes a blood flow sensor, adapted to measure a rate of blood flow within the hepatic portal vein. For some applications, the blood flow sensor includes a blood flow meter. Alternatively or additionally, the blood flow sensor includes one or more electrodes, adapted to be placed on an external surface of the portal vein, and the blood flow sensor is adapted to measure a change in impedance between the electrodes.

There is still further provided, in accordance with an embodiment of the present invention, apparatus including:

one or more electrodes, adapted to sense an electrical signal generated by a portoarterial glucose sensor of the subject; and

a control unit, adapted to:
analyze the signal, and
responsive to the analysis, detect an indication of a blood glucose concentration of the subject.

There is additionally provided, in accordance with an embodiment of the present invention, apparatus including an electrode, which includes a wire shaped so as to define an arc of between about 270 and about 359 degrees, the electrode adapted to be applied around an external surface of a vein.

In an embodiment, the vein includes a hepatic portal vein, and the electrode is adapted to be applied around the external surface of the portal vein.

For some applications, the electrode includes an electrically insulating coating that coats a portion of a circumference of the wire that does not come in contact with the external surface of the vein.

For some applications, the electrode includes a removable curved needle, adapted to draw the electrode around the surface of the vein beneath at least a portion of connective tissue surrounding the vein, and be removed after completion of the drawing.

There is yet additionally provided, in accordance with an embodiment of the present invention, apparatus including:

a support structure; and

two or more electrodes coupled to the support structure, each of the electrodes including a wire shaped so as to define an arc of between about 270 and about 359 degrees, the electrodes adapted to be applied around an external surface of a vein in an embodiment, the vein includes a hepatic portal vein, and the electrodes are adapted to be applied around the external surface of the portal vein.

For some applications, each of the electrodes includes an electrically insulating coating that coats a portion of a circumference of the wire that does not come in contact with the external surface of the vein.

For some applications, each of the electrodes includes a removable curved needle, adapted to draw the electrode around the surface of the vein beneath at least a portion of connective tissue surrounding the vein, and be removed after completion of the drawing.

For some applications, the support structure includes an elongated rod. For some applications, the rod has a length of between about 2 and about 3 cm. For some applications, the rod has a length of between about 1 and about 3 mm.

There is also provided, in accordance with an embodiment of the present invention, apparatus including:

an electrode lead having a distal region and a proximal end, the electrode lead adapted to be placed in a hepatic portal vein of a subject by being advanced through: (a) an opening in a mesenteric vein of the subject that empties into a superior mesenteric vein of the subject, (b) the mesenteric vein, and (c) the superior mesenteric vein, until a distal region of the electrode reaches the portal vein; and

a control unit, adapted to be coupled to the proximal end of the electrode lead, and to perform at least one action selected from the list consisting of: driving the electrode lead to apply an electrical current to the portal vein, and sensing electrical activity of the portal vein through the electrode lead.

For some applications, a portion of the electrode lead includes an electrically insulating coating except at the distal region of the electrode lead.

For some applications, the apparatus includes an external electrode, adapted to be placed external to the portal vein in a vicinity thereof, and the control unit is adapted to be coupled to the external electrode.

The present invention will be more fully understood from the following detailed description of embodiments thereof, taken together with the drawings, in which:

### BRIEF DESCRIPTION OF THE DRAWINGS

FIG. 1 is a schematic illustration of a hepatic interface system applied to a human liver, in accordance with an embodiment of the present invention;

FIG. 2 is a schematic block diagram of another hepatic interface system applied to a nerve of a subject, in accordance with an embodiment of the present invention;

FIG. 3 is a schematic illustration of an electrode device, in accordance with an embodiment of the present invention;

FIG. 4 is a schematic cross-sectional view of one of the electrodes of the device of FIG. 3, in accordance with an embodiment of the present invention;

FIGS. 5A, 5B, and 5C are schematic illustrations of an electrode, in accordance with an embodiment of the present invention; and

FIG. 6 is a schematic illustration of another electrode implanted in a portal vein of a subject, in accordance with an embodiment of the present invention.

### DETAILED DESCRIPTION OF EMBODIMENTS

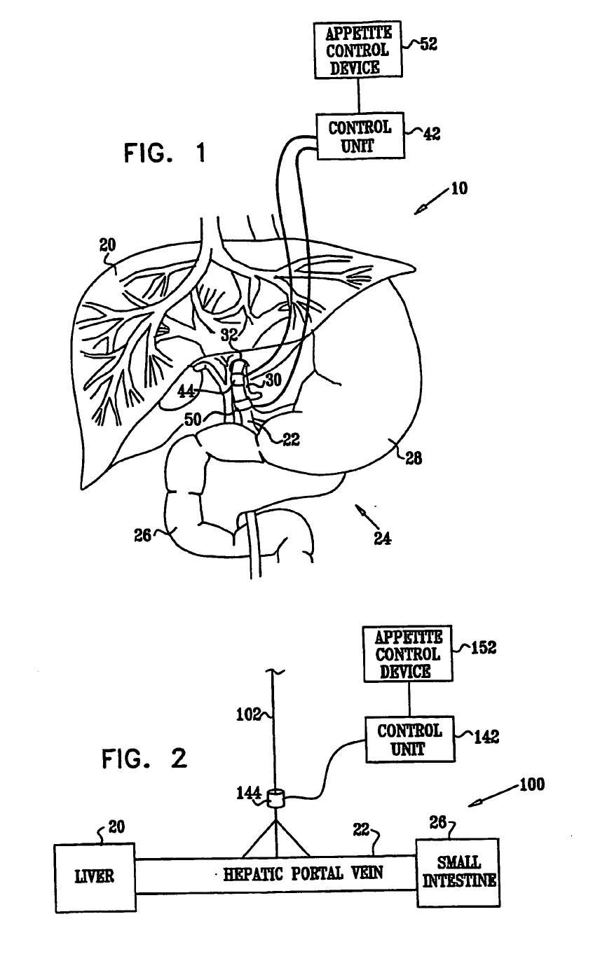

FIG. 1 is a schematic illustration of a hepatic interface system 10 applied to tissue of or associated with a human liver 20, in accordance with an embodiment of the present invention. A hepatic portal vein 22 carries blood to liver 20 from a digestive tract 24, including a small intestine 26 and a stomach 28. A hepatic artery 30 brings oxygen-rich blood to liver 20. Portal vein 22 and hepatic artery 30 enter liver 20 through a hepatic hilum 32.

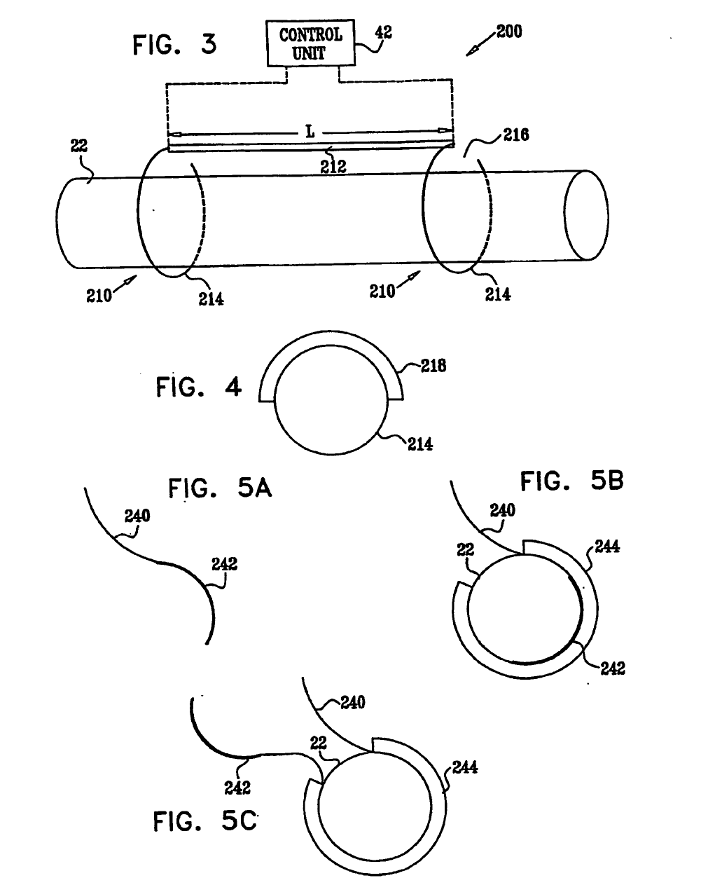

System 10 comprises an implantable or external control unit 42 coupled to an electrode device 44. Electrode device 44 typically comprises one or more electrodes that are either placed in portal vein 22, for example as described hereinbelow with reference to FIG. 4, or applied to an external surface of portal vein 22. In the latter case, the electrodes typically comprise cuff electrodes or wire electrodes, for example as described hereinbelow with reference to FIG. 3. Electrode device 44 is generally applied to portal vein 22 in a vicinity of hepatic hilum 32.

In an embodiment of the present invention, control unit 42 drives electrode device 44 to apply an electrical current to portal vein 22. The control unit typically configures the current to stimulate afferent nerve terminals embedded in the wall of portal vein 22 and/or in liver 20. Additionally, the control unit typically configures the current to generally minimize artificially-induced activation of muscle tissue of the vein. Depending on the specific design of the electrodes and electrode device 44, appropriate parameters of the current may include a frequency of between about 0.1 Hz and about 5 Hz (e.g., 1 Hz), between about 5 Hz and about 100 Hz (e.g., 20 Hz), or between about 100 Hz and about 1 kHz (e.g., 200 Hz), and an amplitude of between about 0.1 milliamp and about 1 milliamp (e.g., 3 milliamps), between about 1 milliamp and about 15 milliamps (e.g., 4 milliamps), or between about 15 milliamps and about 20 milliamps.

Such electrical stimulation generally activates the portoarterial glucose sensor, in a manner similar to the natural activation of this physiological sensor by a positive glucose gradient between portal vein 22 and hepatic artery 30, as described hereinabove. Such stimulation thus generally increases uptake of blood glucose, e.g., by muscle, liver, and/or adipose tissue, thereby normalizing postprandial hyperglycemia, substantially lowering inter-meal blood glucose levels, and/or reducing food intake of the subject. Such stimulation therefore is generally useful for treating subjects suffering from various medical conditions, such as obesity, diabetes, and/or heart disease, or for preventively treating subjects considered at risk of developing such conditions.

In some cases, portal vein electrical stimulation increases uptake of blood glucose by a physiological mechanism not mediated by insulin. In these cases, uptake is increased even in the absence of an increase in blood insulin concentration. Thus, the stimulation is generally effective even for patients suffering from insulin resistance, such as those suffering from type II diabetes. Alternatively, the stimulation increases uptake of blood glucose by a physiological mechanism mediated by insulin, typically by an increase in blood insulin concentration.

In an embodiment of the present invention, portal vein electrical stimulation is applied at least intermittently on a long-term basis, e.g., for greater than 3 days, or for greater than one month. For some patients, such long-term stimulation reduces the amount of insulin secreted by the patient's pancreas, thereby reducing beta-cell exhaustion and allowing the pancreas to recuperate over time. In addition, a reduction in beta-cell exhaustion may provide more time for proinsulin to mature to insulin in the pancreas, thereby allowing the pancreas to increase the ratio of excreted insulin to proinsulin, relative to the same ratio in the absence of the stimulation.

In an embodiment of the present invention, control unit 42 uses electrode device 44 to sense electrical changes in activity of portal vein 22. Such sensing is typically configured to sense electrical activity indicative of activity of afferent nerve fibers that innervate portal vein 22. Such sensing is thus generally indicative of the level of activation of the portoarterial glucose sensor of the subject, and is therefore generally correlated with the quantity of glucose recently absorbed by the small intestine during eating. In this embodiment, therefore, system 10 serves as an indicator of eating. For some applications, a type of electrical change which is detected is a decrease in electrical activity in the portal vein. Other changes include, alternatively or additionally, increases in electrical activity, changes in heights of different measured peaks, and/or changes in the relative timing of different peaks in the sensed electrical activity.

In an embodiment of the present invention, system 10 alternatively or additionally comprises a food intake detection device 50, which is coupled to portal vein 22. For some applications, food intake detection device 50 comprises a blood flow sensor, which is adapted to detect a rate of blood flow in portal vein 22. Increased portal blood flow is generally indicative of elevated postprandial portal blood glucose, carbohydrate, fat, or protein levels, and thus indicates the quantity of such products recently absorbed by small intestine 26 during eating. For example, the blood flow sensor may comprise an implantable blood flow meter, e.g., a pulsed Doppler ultrasonic flow meter, or a meter that utilizes the thermodilution principle. Alternatively, the blood flow sensor comprises one or more electrodes adapted to measure changes in impedance between the electrodes, which changes are indicative of stretching of portal vein 22 caused by increased portal blood flow. For such applications, detection device 50 may comprise electrode device 44, a separate electrode device similar to electrode device 44, or other separate electrodes. In an embodiment, detection device 50 uses chemical or non-chemical blood analysis techniques to directly detect glucose levels. For example, the glucose detection device may utilize: (a) near-infrared or infrared absorption spectroscopy, or (b) a laser transducer implanted on the portal vein together with an acoustic sensor that measures changes in acoustic reflections from the blood, which reflections are correlated with blood glucose level. Alternatively, the glucose detection device uses more invasive techniques, such as those described in the above-mentioned U.S. Pat. No. 6,514,718 to Heller et al. or the above-mentioned U.S. Pat. No. 5,368,028 to Palti.

In an embodiment of the present invention, control unit 42 is configured to drive electrode device 44 to stimulate portal vein 22 responsive to detection of eating by the subject. For some applications, such eating detection is performed using one or more of the techniques described hereinabove. Alternatively, such detection is performed using techniques described in the above-mentioned '953 patent, the above-mentioned '968 PCT publication, the above-mentioned US Provisional Patent Application, entitled, “Gastrointestinal methods and apparatus for use in treating disorders,” or using techniques known in the art. In another embodiment of the present invention, control unit 42 is configured to drive electrode device 44 to stimulate portal vein 22 generally constantly, not responsive to detection of eating. Alternatively, the stimulation is applied periodically, such as during certain times of day or night, or in response to a command from the subject.

In an embodiment of the present invention, system 10 comprises an appetite control device 52. Upon detection of eating using the techniques described herein, control unit 42 drives appetite control device 52 to reduce an appetite of the subject, such as by increasing a sensation of satiety. For example, appetite control device 52 may utilize appetite reduction techniques (a) described in the above-mentioned '953 patent and/or the above-mentioned '968 PCT publication, (b) described in the gastric band or balloon patents mentioned in the Background of the Invention, and/or (c) known in the art (e.g., by administration of an appetite suppressing medication). Alternatively or additionally, upon detection of eating using the techniques described herein, system 10 performs the colonic stimulation techniques described in the above-mentioned US Provisional Patent Application, entitled, “Gastrointestinal methods and apparatus for use in treating disorders.”

In an embodiment of the present invention, upon detection of an elevated blood glucose level, such as by using the techniques described herein, control unit 42 drives an internal or implanted device to increase insulin levels in blood of the subject. For example, devices appropriate for increasing insulin levels include, but are not limited to:
insulin pumps, as known in the art;
devices for increasing insulin secretion by direct or indirect stimulation of the pancreas, such as those described in US Patent Application Publication 2003/0208242 to Harel et al.; PCT Patent Publication WO 03/45493 to Harel et al.; and/or U.S. Pat. No. 5,919,216 to Houben et al., all of which are incorporated herein by reference; and
devices for modulating insulin secretion by direct stimulation of the vagus nerve, such as those described in the above-referenced U.S. Pat. Nos. 5,188,104, 5,231,988, and/or 5,263,480 to Wernicke et al.
   For some applications, elevated glucose levels are alternatively or additionally treated using conventional pharmaceutical approaches.

Reference is now made to FIG. 2, which is a schematic block diagram of a hepatic interface system 100 applied to a nerve 102 of a subject, in accordance with an embodiment of the present invention. System 100 is generally similar to hepatic interface system 10, as described hereinabove with reference to FIG. 1, except for differences described hereinbelow. System 100 comprises a control unit 142, and, for some applications, an appetite control device 152. In addition, system 100 comprises an electrode device 144, adapted to be coupled to nerve 102. Nerve 102 conducts afferent impulses generated by the portoarterial glucose sensor. For example, nerve 102 may comprise a vagus nerve of the subject, or a branch of the vagus nerve, such as a hepatic branch of the vagus nerve. To detect activation of the portoarterial glucose sensor, control unit 142 senses electrical activity of nerve 102, using, for example, techniques described hereinabove with reference to FIG. 1, mutatis mutandis. To effect appetite suppression, control unit 142 drives electrode device 144 to apply a current to nerve 102, with parameters generally configured to mimic the natural afferent nerve signals generated by the portoarterial glucose sensor. For example, the current may be configured to be such as to excite affected tissue, or to inhibit affected tissue.

Reference is now made to FIG. 3, which is a schematic illustration of an electrode device 200, in accordance with an embodiment of the present invention. For some applications, electrode device 44 and/or detection device 50 comprises electrode device 200. Electrode device 200 typically comprises two or more electrodes 210 coupled to a support structure 212, which typically comprises an elongated rod. Alternatively, electrode device 200 comprises a single electrode 210. When electrode device 200 is used for stimulation, support structure 212 typically has a length L of between about 2 cm and about 3 cm. When the electrode device is used for sensing, length L is typically between about 1 mm and about 3 mm.

Typically, each of electrodes 210 is generally circular in shape, and comprises a flexible wire 214, comprising, for example, titanium nitride or IrO2. Electrodes 210 are adapted to be applied to an external surface of portal vein 22. The electrodes are typically shaped so as to surround all or a portion of the vein. For example, each of the electrodes may be shaped so as to define an arc of between about 270 and about 359 degrees, with an opening 216 of between about 1 and about 90 degrees. Opening 216 and the flexibility of wire 214 generally prevent the electrodes from constricting blood flow in vein 22. For some applications, electrodes 210 are helical in shape.

Reference is now made to FIG. 4, which is a schematic cross-sectional view of one of electrodes 210, in accordance with an embodiment of the present invention. In this embodiment, wire 214 comprises an electrically insulating coating 218 that coats a portion of a circumference of the wire that does not come in contact with the external surface of portal vein 22. Alternatively, wire 214 is not electrically insulated (configuration not shown in FIG. 4).

For some applications, electrode device 44 is adapted to be applied to a vein other than portal vein 22, for electrical stimulation and/or sensing of the vein.

Reference is now made to FIGS. 5A, 5B, and 5C, which are schematic illustrations of an electrode 240, in accordance with an embodiment of the present invention. For some applications, electrode 210 of FIG. 3 comprises electrode 240. Alternatively, electrode 240 is not coupled to a support structure like support structure 212. Electrode 240 comprises a removable curved needle 242. During an implantation procedure, a surgeon uses the needle to draw electrode 240 around portal vein 22 beneath connective tissue 244 surrounding the vein, as shown in FIG. 5B. After surrounding a desired portion of the vein, the surgeon removes the needle from the connective tissue, and then separates the needle from electrode 240, leaving the electrode in place around the vein, as shown in FIG. 5C.

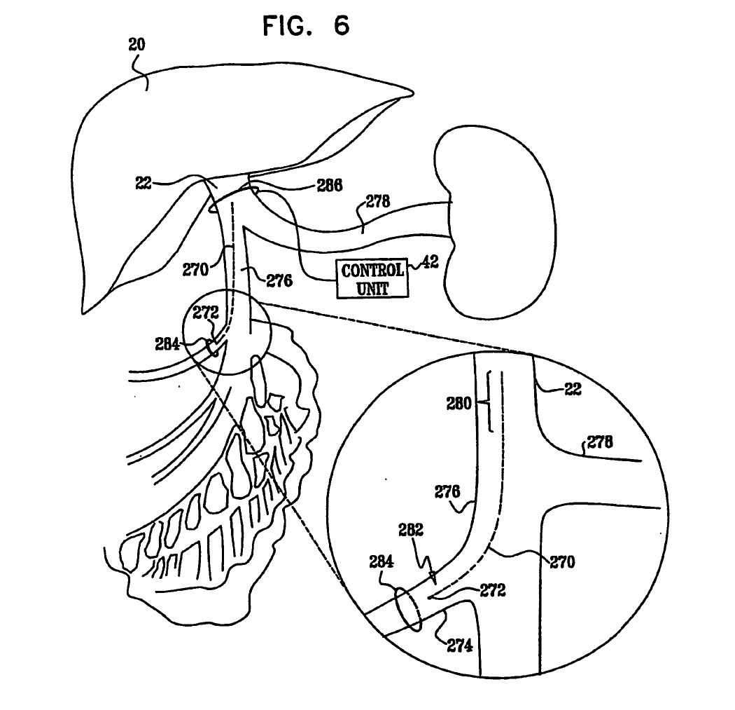

Reference is now made to FIG. 6, which is a schematic illustration of an electrode lead 270 placed in portal vein 22, in accordance with an embodiment of the present invention. For some applications, electrode device 44 and/or detection device SO comprises at least one electrode lead 270. Electrode lead 270 is adapted to be placed in portal vein 22 by making an opening 272 in a mesenteric vein 274 that empties into a superior mesenteric vein 276 (which in turn joins a splenic vein 278 to form portal vein 22). Electrode lead 270 is inserted through opening 272 and advanced until a distal region 280 of the electrode reaches portal vein 22. A proximal end 282 of electrode lead 270 is then secured to mesenteric vein 274 in a vicinity of opening 272. Typically, mesenteric vein 274 is tied off at a site 284 upstream from opening 272, so that blood that would otherwise flow through mesenteric vein 274 does not dislodge electrode lead 270. Typically, the portion of electrode lead 270 placed in the vein comprises an electrically insulating coating except at distal region 280 thereof.

For some applications, at least one external electrode 286, such as electrode 210 (FIG. 3), is additionally applied to the surface of portal vein 22. Control unit 42 drives a current between, and/or senses electrical activity between, electrode lead 270 and external electrode 286. Alternatively, another electrode is provided in a vicinity of electrode lead 270, such as an electrode integrated with a case of the control unit (configurations not shown).

For some applications, hepatic interface system 10 or 100 is implemented as a closed loop system for the treatment of a condition, such at type II diabetes. The control unit determines blood glucose level by sensing portal vein electrical activity and/or vagal activity, as described hereinabove. Responsive to the sensed blood glucose level, the control unit causes a reduction in the blood glucose level by (a) driving the electrode device to electrically stimulate the portal vein, (b) driving electrodes to electrically stimulate the pancreas, such as by using techniques described in the above-mentioned '464 patent application, the above-mentioned '183 PCT publication, and/or the above-mentioned '858 PCT publication, and/or (c) using a technique known in the art.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-75. (canceled)

76. A method for stimulating a vein of a subject, comprising:
placing, around an external surface of the vein, a wire shaped so as to define an arc of between about 270 and about 359 degrees; and
driving an electrical current through the wire, so as to stimulate the vein.

77. The method according to claim 76, wherein the vein includes a hepatic portal vein, and wherein placing the wire comprises placing the wire around the external surface of the portal vein.

78. The method according to claim 76, wherein the electrode includes a removable curved needle, and wherein placing the wire comprises:
using the needle to draw the electrode around the external surface of the vein beneath at least a portion of connective tissue surrounding the vein; and
removing the needle after completion of the drawing.

79. A method for sensing electrical activity of a vein of a subject, comprising:
placing, around an external surface of the vein, a wire shaped so as to define an arc of between about 270 and about 359 degrees; and
sensing the electrical activity of the vein, using the wire.

80. The method according to claim 79, wherein the vein includes a hepatic portal vein, and wherein placing the wire comprises placing the wire around the external surface of the portal vein.

81. The method according to claim 79, wherein the electrode includes a removable curved needle, and wherein placing the wire comprises:
using the needle to draw the electrode around the external surface of the vein beneath at least a portion of connective tissue surrounding the vein; and
removing the needle after completion of the drawing.

82-163. (canceled)

164. Apparatus comprising an electrode, which comprises a wire shaped so as to define an arc of between about 270 and about 359 degrees, the electrode adapted to be applied around an external surface of a vein.

165. The apparatus according to claim 164, wherein the vein includes a hepatic portal vein, and wherein the electrode is adapted to be applied around the external surface of the portal vein.

166. The apparatus according to claim 164, wherein the electrode comprises an electrically insulating coating that coats a portion of a circumference of the wire that does not come in contact with the external surface of the vein.

167. The apparatus according to claim 164, wherein the electrode comprises a removable curved needle, adapted to:
draw the electrode around the surface of the vein beneath at least a portion of connective tissue surrounding the vein, and
be removed after completion of the drawing.

168. Apparatus comprising:
a support structure; and
two or more electrodes coupled to the support structure, each of the electrodes comprising a wire shaped so as to define an arc of between about 270 and about 359 degrees, the electrodes adapted to be applied around an external surface of a vein.

169. The apparatus according to claim 168, wherein the vein includes a hepatic portal vein, and wherein the electrodes are adapted to be applied around the external surface of the portal vein.

170. The apparatus according to claim 168, wherein each of the electrodes comprises an electrically insulating coating that coats a portion of a circumference of the wire that does not come in contact with the external surface of the vein.

171. The apparatus according to claim 168, wherein each of the electrodes comprises a removable curved needle, adapted to:
draw the electrode around the surface of the vein beneath at least a portion of connective tissue surrounding the vein, and
be removed after completion of the drawing.

172. The apparatus according to any one of claims 168-171, wherein the support structure comprises an elongated rod.

173. The apparatus according to claim 172, wherein the rod has a length of between about 2 and about 3 cm.

174. The apparatus according to claim 172, wherein the rod has a length of between about 1 and about 3 mm.

175-177. (canceled)
